(19) United States
(12) Plant Patent Application Publication
(10) Pub. No.: US 2017/0311497 P1
(43) Pub. Date: Oct. 26, 2017

(54) Rose plant named 'RNF Pink 01'

(50) ;

(71) Applicant: Kaneya Co., Ltd., Chita (JP)

(72) Inventor: Hitoshi Nakashima, Gifu (JP)

(21) Appl. No.: 14/999,305

(22) Filed: Apr. 21, 2016

(51) Int. Cl. A01H 5/02

(52) U.S. Cl. CPC A01H 5/0222
USPC PLT/107

(57) ABSTRACT

A new and distinct rose plant having a strong fragrance and deep pink-colored flowers.

Botanical classification: Rosa L.

Varietal denomination: ‘RNF Pink 01’.

### BACKGROUND OF THE INVENTION

The present invention comprises a new and distinct cultivar of rose plant known by the varietal name ‘RNF Pink 01’. The new variety was discovered in April of 2010 in Gifu, Japan. The new variety is the result of a planned breeding program between a Rosa L. variety named ‘Bridal Pink’ (female parent, U.S. Plant Pat. No. 2,851) and an unnamed and unpatented Rosa Rugosa variety (male parent) from the breeder's own collection. ‘RNF Pink 01’ is similar to its female parent in thorn shape and upright growth habit, but ‘RNF Pink 01’ has a deeper pink flower color, fewer thorns, and more buds per stem that bloom like a bunch that differentiate it from its female parent. Additionally, ‘RNF Pink 01’ exhibits a similar petal shape to its male parent, but differs from its male parent in its actual fragrance and the fact that ‘RNF Pink 01’ has an upright growth habit versus its male parent's spreading growth habit. When ‘RNF Pink 01’ is compared to Rosa L. ‘Evera 186’ (currently unpatented in the U.S., Japanese Patent No. 20884), the varieties are similar in that they both exhibit an upright growth habit and double flowers, but ‘RNF Pink 01’ has a slightly lighter pink flower color and different petal shape than ‘Evera 186’. When generally compared to other rose varieties known to the breeder, the new variety differs in its strong fragrance, its type of fragrance, and in flower color. ‘RNF Pink 01’ has been trial and field tested and has been found to retain its distinctive characteristics and remain true to type through successive propagations.

### DESCRIPTION OF THE DRAWINGS

The accompanying photographic drawings illustrate the new variety, with the color being as nearly true as is possible with color illustrations of this type:

FIG. 1 shows a close-up view of a flower of the new variety;

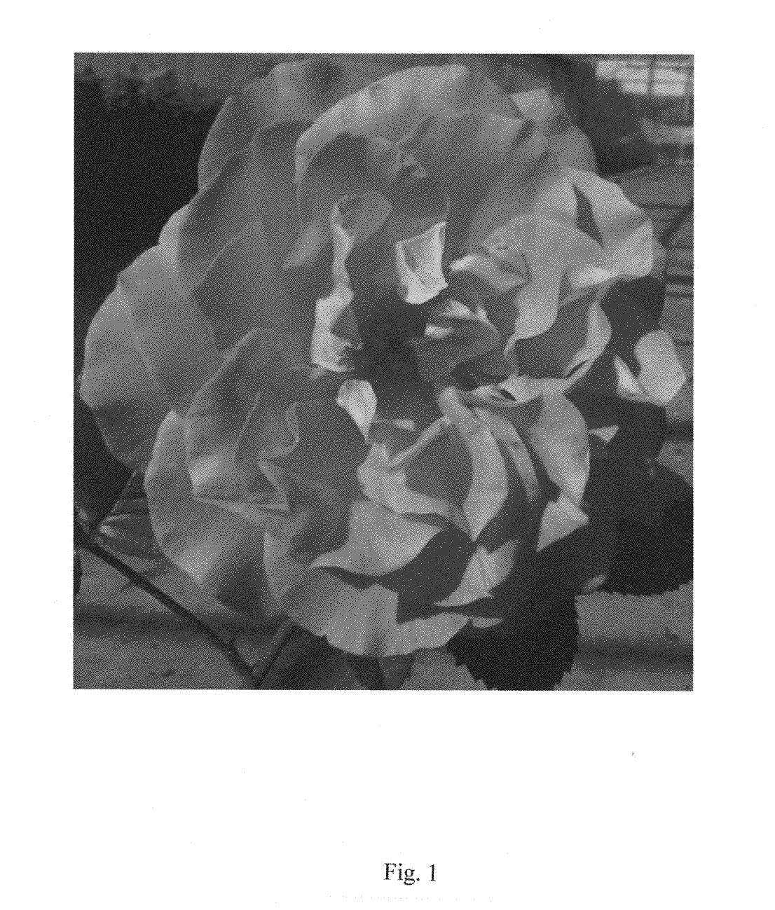

FIG. 2 shows a view of multiple plants of the new variety;

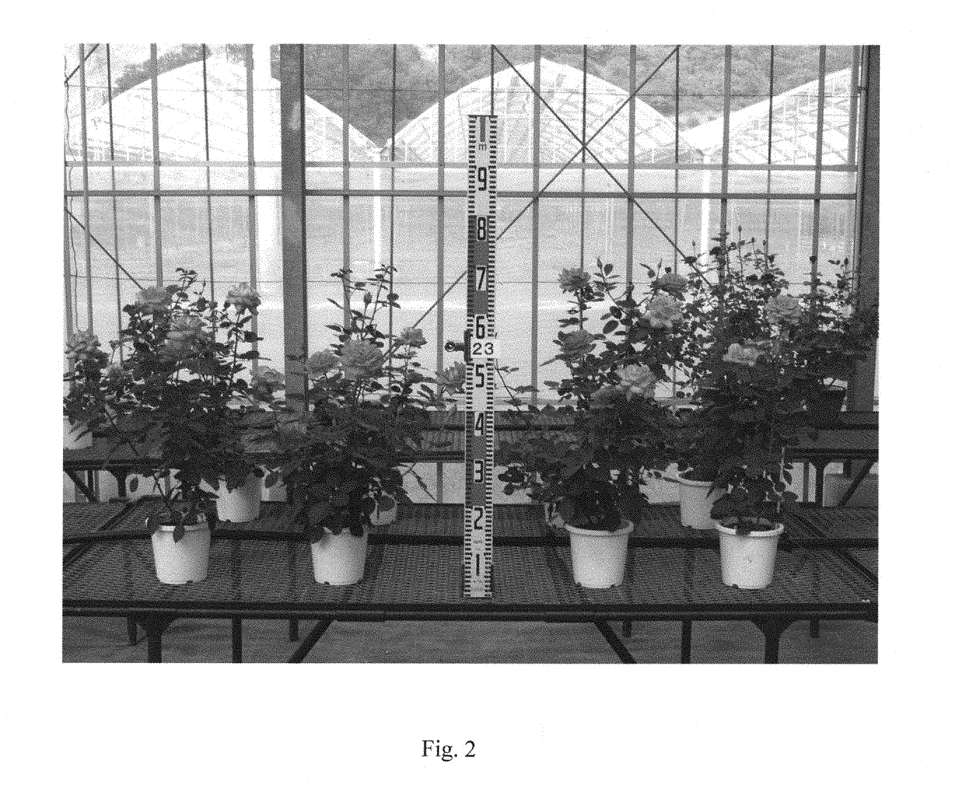

FIG. 3 shows a view of multiple plants of the new variety; and

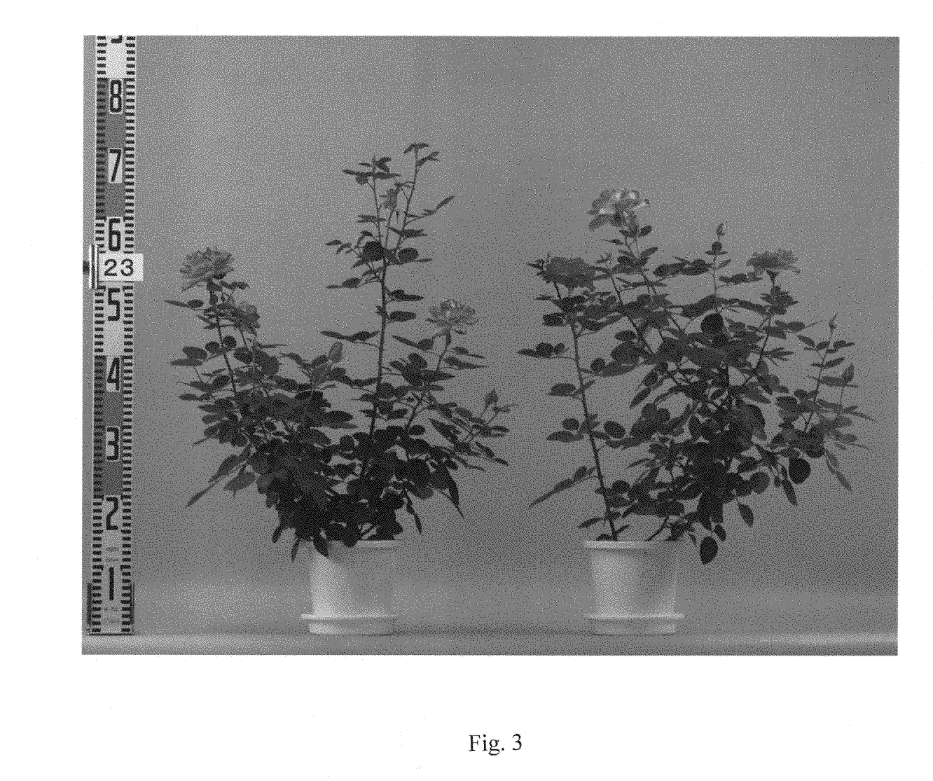

FIG. 4 shows the leaves of the new variety.

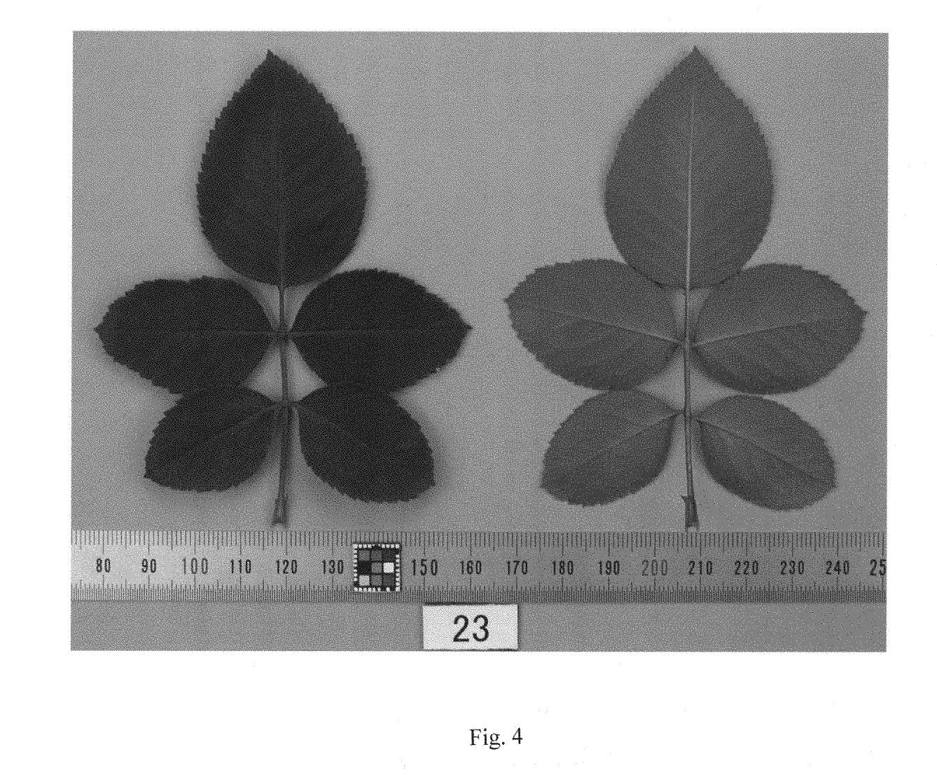

### DESCRIPTION OF THE PLANT

The following detailed description sets forth the characteristics of the new cultivar. The data which defines these characteristics were collected by asexual reproductions first performed via cuttings in May of 2011 carried out in Gifu, Japan. Plants of the new variety were grown in a greenhouse under natural light in 15 cm pots with peat moss substrate and with the assistance of heaters in the winter. The color readings and measurements were taken indoors in a greenhouse under natural light on one-year-old plants grown in containers. Color references are primarily to the R.H.S. Colour Chart of The Royal Horticultural Society of London.
Plant:
   Time to initiate roots.—About 21-28 days at approximately 25° C.
   Time to develop roots.—About 30-45 days at approximately 25° C.
   Time to produce a finished flowering plant from a rooted cutting.—About 5 months/20-22 weeks in a 15 cm container.
   Rooting habit.—Similar to standard roses.
   Form.—Upright.
   Distance from media surface to top of foliage.—30-35 cm at the end of production.
   Distance from media surface to top of flowers.—35-40 cm.
   Plant diameter.—Around 25 cm.
   Vigor.—Average.
   Strength (need for artificial support).—No artificial support needed.
   Branching characteristics.—Branching from bottom or lower level.
   Stem color.—Immature: N57A. Mature: 135D.
   Surface texture.—Young wood: Similar to standard roses. Older wood: Similar to standard roses.
   Average stem diameter.—From 7 mm to 1 cm.
   Internode length.—Approximately 4 to 5 cm.
   Number of internodes.—6 to 7.
   Thorns.—Average number: Main branches: 70 per plant. Stems: 12 per stem. Length: 7 mm to 1 cm. Width: Around 5 mm. Color: Ivory/beige to red (65A). Shape: Similar to standard roses.
   Foliage.—Arrangement: Alternate. Average number of leaves per lateral branch: 5. Average number of leaflets per leaf: 5. Overall size of leaf: Length: 10 cm. Width: 5 cm. Leaflet: Shape of leaf (generally): Ovate and similar to standard roses. Shape of apex: Acute and similar to standard roses. Shape of base: Obtuse and similar to standard roses. Length range: 5-7 cm. Width range: 3-5 cm. Texture: Similar to standard roses. Margin type: Serrate and similar to standard roses. Color: Young leaves: Upper surface: 141C. Lower surface: 144C. Mature leaves: Upper surface: 132A. Lower surface: 135D. Veins: Venation type: Pinnate and similar to standard roses. Color: 135D. Petiole: Length: 2-3 mm. Diameter: 1-2 mm. Color: 135C with a hint of red. Texture: Similar to standard roses. Stipule: None present.
Inflorescence:
   Buds.—Number of buds per plant: 10. Number of buds per flowering stem: 2. Shape of individual bud: Napiform and similar to standard roses. Length of individual bud: 1.5-2 cm. Width of individual bud: 8 mm to 1 cm. Bud color: Green with red.
   Natural flowering season at specified location.—End of April to May in Gifu, Japan.
   Average number of flowers per year at specified location.—10 to 15.
   Flowering habit.—Continuous and freely flowering.
   Flower description.—2 or more flowers and bloom like a bunch.
   Lastingness of an individual flower on the plant.—10 days to 2 weeks.
   Lastingness of a cut flower.—1 week.
   Flower.—General appearance: Double flowering rose. Shape: Round and mounded. Diameter: 8-10 cm. Height: 3-4 cm.
   Petals.—Color: Upper surface (when opening): 67C. Lower surface (when opening): 67C with a bit of white. Upper and lower surfaces (fully opened): 67C. Shape: Rounded. Length: 4-5 cm. Width: 5-6 cm. Apex shape: Round with a wide lip. Base shape: Round and narrow. Margin: Entire. Number per flower: 25-35. Texture: Soft and similar to standard rose petals. Reflex characteristics: None. Presence of basal petal spots: None. Petaloid description: Lightly fringed with semi-double flowering.
   Sepals.—Color: Upper and lower surfaces (when opening): Green. Upper and lower surfaces (fully opened): Green. Shape: Sharp. Length: 2-3 cm. Width: 8 mm to 1 cm. Apex shape: Pointed. Base shape: Wide. Margin: Entire and tight, when closed. Number per flower: 4-5. Texture: Similar to standard roses.
   Pedicel.—Length: 3-5 cm. Diameter: 3 mm. Texture: Similar to standard roses. Strength: Strong. Color: 141D.
   Peduncle color.—141D.
   Receptacle.—Texture: Same as a standard Floribunda-type rose. Shape: Same as a standard Floribunda-type rose. Height: 5-7 mm. Width: 5-7 mm. Color: Green.
   Fragrance.—Strong.
   Disease/pest resistance.—Average, nothing unusual noted to date.
   Winter temperature tolerance.—Blooming at 1° C. in winter. With only roots, −15˜−-18° C.
   Hot temperature tolerance.—40° C.
   Drought tolerance.—Average.
   Reproductive organs.—
   Stamens.—Number (per flower): 50.Filament: Length: 7-10 mm. Color: White. Anthers: Shape: Like a match stick. Length: 2-3 mm. Color: Light yellow/beige. Pollen: Color: 12D. Amount (generally): Less than average.
   Pistils.—Number: 50. Length: 1-2 mm. Style: Length: Average. Color: Creamy yellow. Stigma: Shape: Similar to standard roses. Color: Creamy yellow/beige. Size: 1 mm. Ovaries: Length: 3 mm. Width: 0.5 mm. Color: White.

I claim:

1. A new and distinct variety of rose plant, as is herein illustrated and described.
